(19) United States
(12) Patent Application Publication
(10) Pub. No.: US 2003/0216413 A1
(43) Pub. Date: Nov. 20, 2003

(54) Catecholamine pharmaceutical compositions and methods

(76) Inventors: Robert S. Root-Bernstein, East Lansing, MI (US); Patrick F. Dillon, East Lansing, MI (US)

Correspondence Address: HARNESS, DICKEY & PIERCE, P.L.C., P.O. BOX 828, BLOOMFIELD HILLS, MI, 48303, US

(21) Appl. No.: 10/401,421

(22) Filed: Mar. 28, 2003

Related U.S. Application Data
(63) Continuation-in-part of application No. PCT/US01/30272, filed on Sep. 27, 2001.
(60) Provisional application No. 60/236,751, filed on Sep. 29, 2000.

(51) Int. Cl. A61K 31/522; A61K 31/485; A61K 31/198; A61K 31/375; A61K 31/137

(52) U.S. Cl. 514/263.31; 514/282; 514/474; 514/567; 514/649; 514/566

(57) ABSTRACT

Pharmaceutical compositions and method using adrenergic compounds and complement compounds. Compositions are provided comprising:

(c) a subefficacious amount of an adrenergic compound; and

(d) a safe and effective amount of a complement to the adrenergic compound.

Methods are also provided comprising the administration of:

(c) a low dose of an adrenergic compound; and

(d) a safe and effective amount of a complement to said adrenergic compound.

Preferably, the adrenergic compound is a catecholamine. Complements include ascorbates, opioids, polycarboxylic acid chelaters, and mixtures thereof. Preferred complements include ascorbates, particularly ascorbic acid. Methods include the treatment of neurological disorders, hypotension, forward failure, backward failure, congestive heart failure, shock, hypertension, hemorrhage, disorders associated with anesthesia, chronic obstructive pulmonary disease, asthma, colic, Crohn's disease, anaphylaxis, interstitial cystitis, overactive bladder syndrome, premature labor, myethsenia gravis, and glaucoma.

### CROSS-REFERENCE TO RELATED APPLICATIONS

This application is a continuation-in-part of International Application PCT/US01/30272, with an international filing date of Sep. 27, 2001, published in English under PCT Article 21(2), which claims the benefit of U.S. Provisional Application No. 60/236,751, filed Sep. 29, 2000.

### BACKGROUND OF THE INVENTION

This invention relates to novel methods of treating disorders mediated by adrenergic receptors, and novel pharmaceutical compositions containing catecholamines or other adrenergic compounds. For example, the compositions and methods of this invention comprise the use of catecholamines and ascorbates in the treatment of a variety of disorders, including asthma, hypertension, and congestive heart failure.

Catecholamines and related adrenergic (sympathomimetic) drugs are involved in the regulation of a wide variety of body functions. Such compounds have their effect directly or indirectly on the alpha- and beta-adrenergic receptors found in tissues throughout the body. Because the functions that are mediated by these receptors are diverse, agents that agonize or antagonize their activity are useful in the treatment of a variety of clinical disorders.

Most of the actions of adrenergic compounds can be classified into seven broad types: (1) peripheral exciatory action on certain types of smooth muscle, such as those in blood vessels supplying skin and mucous membranes, and on gland cells, such as those in salivary and sweat glands; (2) peripheral inhibitory action on certain other types of smooth muscle, such as those in the wall of the gut, in the bronchial tree, and in blood vessels supplying skeletal muscle; (3) cardiac exciatory action, responsible for an increase in heart rate and force of contraction; (4) metabolic action such as an increase in rate of glycogenolysis in liver and muscle, and liberation of free fatty acids from adipose tissue; (5) endocrine action, such as modulation of the secretion of insulin, renin, and pituitary hormones; (6) CNS action, such as respiratory stimulation and, with some adrenergics, an increase in wakefulness, psychomotor activity, and a reduction in appetite; and (7) presynaptic actions, which result in either inhibition or facilitation of the release of neurotransmitters such as norepinephrine and acetylcholine. See, Goodman and Gilman's, The Pharmacological Basis of Therapeutics, 8^{th} Edition (1990). Disorders that can be treated using adrenergic compounds include, for example, hypertension, shock, cardiac arrhythmia, asthma, allergy, cardiac failure and anaphylaxis. The response of a body tissue to an adrenergic compound is dictated not only by the direct affects of the compound but also by the homeostatic responses of the organism.

Accordingly, the clinical use of adrenergic compounds can be complicated, since administration may affect several different body functions. Side effects are not uncommon, and careful selection must be made of the specific adrenergic compound to be used and the dosage level in which it is to be administered.

### SUMMARY OF THE INVENTION

The present invention provides pharmaceutical compositions comprising adrenergic compounds and complement compounds. Embodiments of this invention include compositions comprising:

(a) a subefficacious amount of an adrenergic compound; and

(b) a safe and effective amount of a complement to said adrenergic compound.

Other embodiments include compositions comprising:

(a) a safe and effective amount of an adrenergic compound; and

(b) a complement to said adrenergic compound, selected from the group consisting of a hyperpreserving amount of an ascorbate, a safe and effective amount of an opioid, a hyperpreserving amount of a polycarboxylic acid chelater, and mixtures thereof.

Methods are also provided for regulating an adrenergic receptor in a human or other animal, comprising the administration of:

(a) low dose of an adrenergic compound; and

(b) safe and effective amount of a complement to said adrenergic compound.

Preferably, the adrenergic compound is a catecholamine. Preferred complements include ascorbates, particularly ascorbic acid. Methods include the treatment of neurological disorders, hypotension, forward failure, backward failure, congestive heart failure, shock, hypertension, hemorrhage, disorders associated with anesthesia, chronic obstructive pulmonary disease, asthma, colic, Crohn's disease, anaphylaxis, interstitial cystitis, overactive bladder syndrome, premature labor, myethsenia gravis, and glaucoma.

It has been found that the compositions and methods of this invention are effective for treating a broad range of disorders associated with adrenergic receptors. Use of these methods and compositions afford advantages versus adrenergic compositions and methods among those known in the art, including enhanced efficacy, increase duration of action, reduction of side effects, and dosing flexibility.

### BRIEF DESCRIPTION OF THE DRAWINGS

FIG. 1 depicts an exemplary plot of efficacy versus dosage of adrenergic compound, with and without administration of a complement.

FIG. 2 depicts an exemplary plot of efficacy of adrenergic compound versus time, with and without administration of a complement.

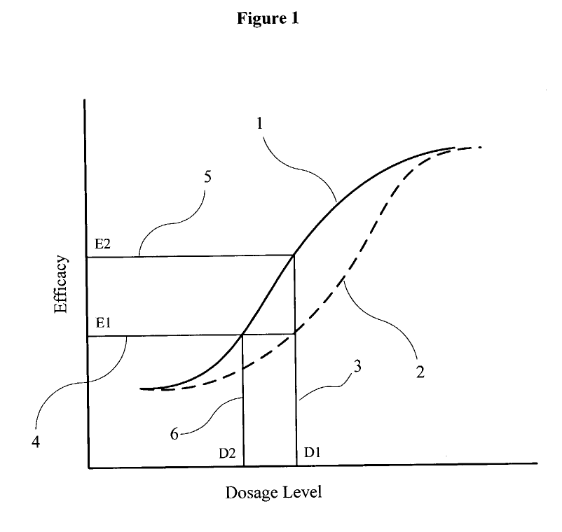

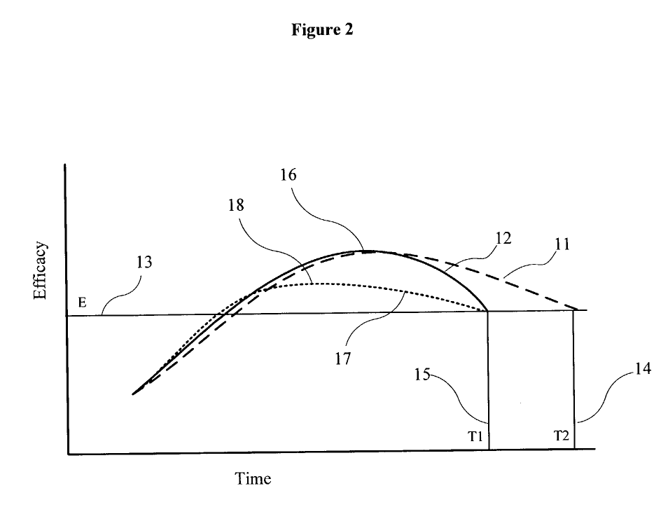

It should be noted that the plots set forth in FIGS. 1 and 2 are intended to show the general characteristics of regimens among those of this invention, for the purpose of the description of such embodiments herein. These plots may not precisely reflect the characteristics of any given embodiment, and are not necessarily intended to define or limit specific embodiments within the scope of this invention.

### DESCRIPTION OF THE INVENTION

The present invention encompasses certain novel compositions and methods for the administration of adrenergic compounds to human or other animal subjects. Specific compounds and compositions to be used in the invention must, accordingly, be pharmaceutically acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

The compositions and methods of this invention preferably comprise the administration of an adrenergic compound and a complement to said adrenergic compound at “synergistic” levels. Accordingly, the therapeutic effect of administering of the combination of the adrenergic compound and complement is greater than the additive effect of administering the adrenergic compound and the complement individually. Such effects include one or more of increasing the effect of the adrenergic compound, increasing the duration of the effect of the adrenergic compound, and making adrenergic compounds effective at dosage levels that would otherwise be ineffective. (As used herein, the word “include,” and its variants, is intended to be non-limiting, such that recitation of items in a list is not to the exclusion of other like items that may also be useful in the materials, compositions and methods of this invention. Also as used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.)

Such effects of embodiments included in this invention are set forth in FIGS. 1 and 2. FIG. 1 depicts the efficacy of an adrenergic compound as a function of dosage level, when administered with a complement (1) and when administered without a complement (2). As shown, for a given dosage of adrenergic compound, D1 (3), the adrenergic effect is enhanced from level E1 (4) to level E2 (5). In a preferred embodiment, dosage D1 is a level which, without the presence of a complement, is subefficacious (i.e., efficacy level E1 is not significantly effective clinically). In another preferred embodiment, level E1 is significantly effective clinically, but that level of efficacy is obtained using a lower dosage level of adrenergic compound, D2 (6).

FIG. 2 depicts the effect of an adrenergic compound as a function of time, with and without administration of a complement. Plots of efficacy versus time are shown for a first therapy (11) of adrenergic compound administered with a complement, and a second therapy (12) of adrenergic compound administered at the same level but without a complement. The first therapy (11) maintains efficacy above a desired level of efficacy E (13) for a period of time T2 (14) that is longer than the time T1 (15) during which the second therapy maintains such levels of efficacy. In some embodiments of this invention, as depicted, the maximum efficacy (16) is essentially identical for administration of the adrenergic with (11) and without (12) the complement. In alternative embodiments, the essentially identical maximum efficacy and enhanced duration (T2) is obtained using a lower dose of adrenergic compound when combined with a complement. That is, in such embodiments, the level of adrenergic compound administered in the first therapy (11) is lower than that administered in the second therapy (12). In an alternative third therapy embodiment (17), the adrenergic compound is administered at a lower level with a complement, resulting in a lower maximum efficacy (18), but remaining at or above the desired level of efficacy E (13) for essentially the same duration of time T1 (15) as the second therapy (12) of the adrenergic compound at the higher level without a complement.

Adrenergic Compounds

The adrenergic compounds useful herein are pharmaceutically acceptable compounds which directly or indirectly agonize or antagonize an alpha- or beta-receptor, eliciting a sympathomimetic response. Many adrenergic compounds are known in the art, including those described in Goodman and Gillman's, The Pharmacological Basis of Therapeutics, 8^{th} Edition (1990) (incorporated by reference herein). Adrenergic compounds useful herein include those selected from the group consisting of albuterol, amantadine, amphetamine, benzephetamine, bitolterol, clonidine, colterol, dextroamphetamine, diethylpropion, dobutamine, dopamine, ephedrine, epinephrine, ethylnorepinephrine, fenfluramine, fenoterol, guanabenz, guanfacine, hydroxyamphetamine, isoetharine, isoproterenol, levodopa, mephenxermine, metaproterenol, metaraninol, methamphetamine, methoxamine, methyldopa, methylphendate, norepinephrine, oxymetazoline, pemoline, phendimetrazine, phenmetrazine, phentermine, phenylephrine, phenylethylamine, phenylpropanolamine, pirbuterol, prenalterol, propylhexedrine, pseudoephedrine, ritodrine, terbutaline, theophylline, tyramine, and derivatives thereof, pharmaceutically acceptable salts and esters thereof, and mixtures thereof. Preferred adrenergic compounds include catecholamines, comprising molecules with a catechol (dihydroxybenzene) moeity. Particularly preferred catecholamines include those selected from the group consisting of albuterol, dopamine, ephedrine, epinephrine, levadopa, norepinephrine, oxymetazoline, phenylephrine, phyenylpropanolamine, pseudoephrine, theophiline, and mixtures thereof.

Adrenergic Compound Complements

The compositions and methods of this invention comprise a compound which is a complement to an adrenergic compound. A preferred “complement” is a compound which, in a given composition or method, binds to the adrenergic compound used in said composition or method. Such “binding” is the formation of a complex through physical-chemical interaction of the complement with the adrenergic compound, through means other than covalent bonding. Such binding is described in the following articles, incorporated by reference herein: Root-Bernstein and Dillon, “Molecular Complementarity I: The Complementarity Theory of the Origin and Evolution of Life.” J. Theoretical Biology 188: 447-449 (1997); and Root-Bernstein, “Catecholamines Bind to Enkephalins, Morphiceptin, and Morphine,” Brain Research Bulletin 18: 509-532 (1987).

Binding between a complement and an adrenergic compound can be demonstrated through any physical, chemical, or immunological technique. Physicochemical methods include nuclear magnetic resonance imaging, ultraviolet or visible light spectroscopy, capillary or other forms of electrophoresis, high pressure liquid and other forms of chromatography, pH titration, and buffering. Chemical methods include procedures that can demonstrate binding such as affinity selection using gels, cellulose, glass, plastic, and/or other bound ligands. Immunological procedures that can demonstrate molecular complementarity include, double antibody diffusion (DAD), double antibody enzyme-linked immunosorption assay (DA-ELISA), in which antibody to the catecholamine (or agonist) and antibody to its potential complements are prepared and tested to determine whether the pairs of antibodies bind to one another.

Preferred complements include those selected from the group consisting of an ascorbate, an opioid, a polycarboxylic acid chelator, and derivatives thereof, pharmaceutically acceptable salts and esters thereof, and mixtures thereof. A “pharmaceutically acceptable salt” is a cationic salt formed at any acidic (e.g., carboxyl) group, or an anionic salt formed at any basic (e.g., amino) group. Many such salts are known in the art, as described in World Patent Publication 87/05297, Johnston et al., published Sep. 11, 1987 (incorporated by reference herein). Preferred cationic salts include the alkali metal salts (such as sodium and potassium), and alkaline earth metal salts (such as magnesium and calcium). Preferred anionic salts include the halides (such as chloride salts). A “pharmaceutically acceptable ester” is an ester that does not essentially interfere with the activity of the compounds used herein, or that is readily metabolized by a human or lower animal subject to yield an active compound.

Ascorbates include ascorbic acid and pharmaceutically derivatives and metabolites thereof. Preferred ascorbates include ascorbic acid, sodium ascorbate, calcium ascorbate, L-ascorbic acid, L-ascorbate, dehydrosoascorbic acid, dehydroascorbate, 2-methyl-ascorbic acid, 2-methyl-ascorbate, ascorbic acid 2-phosphate, ascorbic acid 2-sulfate, calcium L-ascorbate dihydrate, sodium L-ascorbate, ascorbylesters, and mixtures thereof. Ascorbic acid is a particularly preferred ascorbate. Polycarboxylic acid chelators include ethylendiamine tetraacetic acid (EDTA), diethylene triamine pentaacetic acid, pharmaceutically acceptable salts thereof, and mixtures thereof.

As referred to herein, an “opioid” is an opiate, synthetic opioid agonist, synthetic opioid partial agonist, a derivative thereof, pharmaceutically acceptable salt or ester thereof, or a mixture thereof. Preferred opioids include opiates and synthetic opioid agonists. Preferred opioids include alfentanil, apomorphine, benzomorphan, buprenorphine, butorphanol, codeine, dezocine, dihydrocodeine, dihydrocodeinone, diphenoxylate, endorphins (such as Met-enkephalin, Leu-enkephalin, dynorphin A, and dynorphin B), fentanyl, heroin (diacetylmorphine), hydrocodone, hydromorphone, kyotorphin, levorphanol, levomethadyl acetate, loperamide, malbuphine, meptazinol, methadone, meperidine, morphiceptin, morphine, nalbuphine, nalmefene, oxymorphone, oxycodone, pentazocine, propoxyphene, sufentanil, and mixtures thereof. Particularly preferred opioids are selected from the group consisting of alfentanil, apomorphine, benzomorphan, codeine, dihydrocodeine, dihydrocodeinone, diphenoxylate, endorphins (such as Met-enkephalin, Leu-enkephalin, dynorphin A, and dynorphin B), fentanyl, heroin (diacetylmorphine), hydrocodone, hydromorphone, kyotorphin, levorphanol, levomethadyl acetate, loperamide, malbuphine, methadone, meperidine, morphiceptin, morphine, nalmefene, oxymorphone, oxycodone, propoxyphene, sufentanil, and mixtures thereof; more preferably selected from the group consisting of apomorphine, morphiceptin, morphine, Leu-enkephalin, Met-enkephalin, and mixtures thereof.

Pharmaceutical Compositions

The compositions of this invention are preferably provided in unit dosage form. As used herein, a “unit dosage form” is a composition of this invention containing an amount of an adrenergic compound and a complement compound that is suitable for administration to a human or lower animal subject, in a single dose, according to good medical practice.

Adrenergic Compound Dosage:

Compositions useful in the methods of this invention comprise a safe and effective amount of an adrenergic compound and a safe and effective amount of a compound which is a complement to said adrenergic compound. In one embodiment, preferred compositions of this invention comprise a subefficacious amount of an adrenergic compound. A “subefficacious amount” of a given adrenergic compound is an amount which is safe and effective when administered to a human or other animal subject in a composition or method of this invention, but which if administered without a complement to said adrenergic compound would have a clinically insignificant effect. A “safe and effective” amount of an adrenergic compound is an amount that is sufficient to have the desired therapeutic effect in the human or lower animal subject, without undue adverse side effects (such as toxicity, irritation, or allergic response), commensurate with a reasonable benefit/risk ratio when used in the manner of this invention. The specific safe and effective amount of the adrenergic compound will, obviously, vary with such factors as the particular condition being treated, the physical condition of the patient, the nature of concurrent therapy (if any), the specific adrenergic compound used, the specific route of administration and dosage form, the carrier employed, and the desired dosage regimen.

In general, the amount of adrenergic compound in a unit dose composition of this invention is preferably from about 1% to about 90%, preferably from about 10% to about 50%, of the uncomplemented clinically efficacious amount of said adrenergic compound administered on a daily basis, divided by the number of doses of said compound to be given in a day. The “uncomplemented clinically efficacious amount” is that amount which is demonstrated to have a desired therapeutic effect according to good medical practice, without the administration of a complement to said adrenergic compound. Preferably the uncomplemented clinically efficacious amount is that which is demonstrated in the art to have clinical utility in the treatment of the disorder to be treated, preferably through controlled clinical studies, more preferably as approved for commercial marketing. The “number of doses” for a given adrenergic compound is the number of doses necessary to maintain an effective concentration of the compound at the site(s) at which the compound is to have a therapeutic effect. The uncomplemented clinically efficacious amount and number of doses will vary according to the adrenergic compound and its pharmacokinetic characteristics, the disorder to be treated, and the route of administration. Preferably, the amount of adrenergic compound in the compositions of this invention is equal to from about 1% to about 90%, preferably from about 10% to about 50%, of the amount of adrenergic compound in the uncomplemented clinically efficacious compositions of the adrenergic compound that are used in the art.

Adrenergic/Complement Dosage Level Determination Method:

In a preferred embodiment, the amount of adrenergic compound is determined using the following methodology. (As discussed above, other methods may be used, however.) In this method, the dosage level of adrenergic compound is determined by reference to its efficacy in an in vitro smooth muscle contraction model. Specifically, smooth muscle tissue is obtained from the aorta of adult New Zealand white rabbits. Adult rabbits of either sex are relaxed with 55 mg/kg ketamine administered intramuscularly. After fifteen minutes, the rabbits are anesthetized with 50 mg/kg Nembutal (pentobarbital sodium, Abbot Labs) administered intraperitoneally. When the rabbits are unresponsive to toe pinch, the abdomen is opened and the abdominal aorta exposed. The aorta is teased from the vena cava and clamped at both the rostral and caudal ends. The aorta is then removed using surgical scissors and placed in a Physiological Salt Solution at about 4° C. The aortic clamps were removed to induce euthanasia. The Physiological Salt Solution (PSS) contains: NaCl 116 mM; KCl 5.4 mM; NaHCO₃ 19 mM; NaH₂PO₄ 1.1 mM; CaCl₂ 2.5 mM; MgSO₄ 1.2 mM; and glucose 5.6 mM. The PSS is aerated with 95% O₂/5% CO₂ to maintain pH 7.4 and warmed to 37° C. before addition to tissue baths.

Tissue rings are prepared from the aorta using the procedures described in Dillon, P. F., Root-Bernstein, R. S., and Holsworth D. D., “Augmentation of aortic ring contractions by angiotensin II antisense peptide” Hypertension 31; 854-860, 1998 (incorporated by reference herein). Specifically, the aorta is debrided of excess connective tissue, flushed of any remaining blood, and placed in fresh PSS. Aortic rings of 3 mm are cut using a single edge razor blade and the rings placed in fresh PSS. The scissor-cut ends are not used. A pair of stainless steel loops with a flat, straight central section is passed through the lumen of each aortic ring. Upper and lower loops are secured to Plexiglas-stainless steel clamps with stainless steel screws. The lower clamp is attached to a micrometer for length adjustment. The upper clamp is connected to a 50 g force transducer with a gold chain. The force transducers are interfaced with an eight channel signal conditioner and recorder.

The rings are immersed in 20 or 25 ml aerated, jacketed tissue baths, and maintained at 37° C. using a circulator. After mounting, each ring is stretched to 5 g and allowed to stress-relax for 2 hours before activation. If stress-relaxation reaches 0 g, the ring is re-stretched to 2 g and allowed to stress-relax until the passive force is stable. This places the rings at muscle lengths near Lo, the optimal length for force development. The rings have a stretched linear length of approximately 3 to 4 mm.

The tissues are activated with a test material in PSS. Solutions of each test material are prepared fresh on the day of the experiment as a concentrated, refrigerated stock and serially diluted in PSS for each experiment approximately 10 minutes (to allow warming to 37° C.) before each contraction. All components are kept separate prior to the experiment. Individual contractions are generated by replacing PSS in the tissue baths with pre-warmed stimulating PSS with the test material.

An initial K+ contraction is made on each ring prior using a test material. Isomolar high K+-PSS is made by reducing the NaCl concentration to 46 mM and increasing KCl to 75.4 mM. Upon administration of test material, the force of each contraction is recorded. The contraction typically lasts approximately 10 minutes, and is then followed by at least 15 minutes of relaxation in PSS before a following contraction is initiated. Relaxation to baseline force typically takes approximately 10 minutes.

At the conclusion of the experiment, the rings are removed from the baths, blotted dry, and weighed to the nearest 0.1 mg. To minimize error that can be introduced by percentage comparisons in dose-response curves, the contractions are normalized to the weight of the ring (g force/mg tissue).

For comparing two different concentrations of the same adrenergic compound, solutions of both are presented to the smooth muscle according to the above methodology. The force of the contraction generated is measured in grams (g). The tissue is weighed in milligrams (mg). The normalized force of the tissue in g/mg is calculated for the two different doses. The experiment is repeated on several tissues. The data from the different tissues is then averaged, and the mean and standard error for the normalized force for the two concentrations calculated. A t-test is performed comparing the data sets. If the two contractions are performed on the same tissue, a paired t-test is used. If the contractions are done on different tissues, an unpaired t-test is used. A t value is calculated and from this a probability value p is determined (the probability that the two means are not different from one another). If the p value is less than 0.05, there is less than a 0.05 chance that the two contractile forces are the same, and therefore the two doses produce significantly different forces.

For comparing the effect of a complement added to an adrenergic compound, the adrenergic compound, in PSS, is presented to the smooth muscle according to the above methodology, and the contraction force is measured. The complement is then added to the adrenergic compound in PSS, and presented to the smooth muscle, and the force measured. The force of the contractions is measured in grams (g). The tissue is weighed in milligrams (mg). The normalized force of the tissue in g/mg is calculated for the two different doses. The experiment is repeated on several tissues. The data from the different tissues is then averaged, and the mean and standard error for the normalized force for the two concentrations are calculated, as discussed above.

In one embodiment of this invention, the uncomplemented clinically efficacious amount of said adrenergic compound is determined according to the above Dosage Level Determination Method. The subject adrenergic compound is tested at various concentrations to determine the level that is effective in mediating a significant adrenergic response in the absence of a complement. Embodiments included among those using this methodology are as depicted in FIGS. 1 and 2, discussed above. This level is correlated from the in vitro experiment to in vivo levels, using methods known in the art. The subefficacious amount of adrenergic compound is the amount in vivo that corresponds to the amount in vitro that yields efficacy that is at least one standard deviation below the uncomplemented clinically efficacious amount as determined by comparison of the two amounts in the Dosage Level Determination Method, above. For example, in FIG. 1, D2 is one standard deviation lower than D1. Alternatively, the subefficacious amount is two standard deviations below the uncomplemented clinically efficacious amount. For example, in FIG. 1, D2 is alternatively two standard deviations lower than D1.

Complement Compound Dosage:

The compositions of this invention also comprise a safe and effective amount of a complement compound. A “safe and effective amount” of a complement compound is an amount that is sufficient to increase the clinical efficacy of a given adrenergic compound in a human or lower animal subject, without undue adverse side effects (such as toxicity, irritation, or allergic response), commensurate with a reasonable benefit/risk ratio when used in the manner of this invention. The specific “safe and effective amount” of the complement compound will, obviously, vary with such factors as the particular adrenergic compound used, the particular condition being treated, the physical condition of the patient, the duration of treatment, the nature of concurrent therapy (if any), the specific dosage form to be used, the carrier employed, the solubility of the compound therein, and the dosage regimen desired.

Some embodiments of this invention comprise compositions comprise: a safe and effective amount of an adrenergic compound; and a hyperpreserving amount of an ascorbate, a hyperpreserving amount of a polycarboxylic acid chelater, or mixture thereof. Other embodiments comprise: a subefficacious amount of a catecholamine; and a complement to said adrenergic compound, selected from the group consisting of a hyperpreserving amount of an ascorbate, a hyperpreserving amount of a polycarboxylic acid chelater, and mixtures thereof.

As referred to herein, a “hyperpreserving amount” of an ascorbate or a polycarboxylic acid chelator is an amount that is in excess of the amount conventionally used (the “preservative level”) to preserve an adrenergic compound in a dosage form (e.g., to prevent the oxidation of an adrenergic compound in solution). Preferably, the preservative level of the complement is that amount which is demonstrated to protect the adrenergic compound in a clinical dosage form from degradation over a reasonable shelf life (e.g., two years) under typical storage conditions. Preferably the preservative level is that which is demonstrated in the art to have preservative utility in compositions comprising adrenergic compounds, preferably at levels approved for commercial marketing of such products. In such embodiments of this invention, the dosage forms of this invention comprise a concentration of complement at least about 10, preferably at least about 25, preferably at least about 50, preferably at least about 100, preferably at least about 150, preferably about 200, times higher than the concentration of adrenergic compound.

In a preferred embodiment, the preservative level is determined according to the following Antioxidant Effect Method. (As discussed above, other methods may be used, however.) In this method, a solution containing the adrenergic compound (e.g., a catecholamine) is placed in a water-jacketed chamber maintained at 37° C. and the time noted. The solution is aerated with a gas mixture containing a known amount of oxygen and/or other gases. At different times, an aliquot of the solution is taken from the chamber and injected into a capillary electropherograph, which separates compounds based on their charge-to-mass ratio. (The conditions used for measuring catecholamines are known in the art.) A sample is injected at a rate of 7.7 nl/sec for 2 seconds into a 98 cm capillary using vacuum injection. The sample is subjected to a 20 kV-to-ground driving force. The carrier buffer is 25 mM sodium borate at pH 9.4. Catecholamine peaks appear in approximately 8 to 15 minutes at a detection window in the capillary and are measured by the change in absorbance at 195 nm. Oxidation produces a different charge-to-mass ratio in the catecholamines, and the oxidized compounds appear at a different time than the unoxidixed forms. For example, oxidized norepinephrine appears at approximately 8 minutes and unoxidized norepinephrine appears at approximately 9 minutes. The size of the unoxidized peak is measured. The logarithm of the fraction of the oxidized peak remaining is plotted against the time since the solution was first placed in the chamber. From this plot, a slope is calculated. The equation determined in this way is: F=1−e^{-t/τ}; where “F” is the fraction oxidized, “e” is the natural logarithm, “t” is the time since the solution was placed in the chamber, and “τ” is the exponential time constant, where when t=τ and F=63.2% oxidized. The time constant is an inverse measure of the oxidation rate, where an increase in the time constant indicates a decrease in the rate of oxidation.

To determine the effect and preservative level of a complement (e.g., ascorbic acid, “AA,” or other ascorbate) on an adrenergic compound (e.g., a catecholamine), the complement is placed in the solution with the adrenergic compound. The solutions are treated as described above. There is a different anti-oxidant concentration in each solution, but a constant concentration of catecholamine. The oxidation rates are measured in the manner described above and a different value of τ, the oxidation rate of the catecholamine, determined for each concentration of anti-oxidant. There will be a sigmoidal relation between the oxidation rate in the absence of the anti-oxidant (τ₀) and rates with increasing anti-oxidant concentration. The asymptote (τₘₐₓ) approached as the concentration of anti-oxidant increases is determined using a linear least-squares fit of the log of the anti-oxidant concentration plotted against the log of the ratio of the (τₘₐₓ/τ₀), with an iterative value of τₘₐₓ used until the error is minimized. The half-maximal inhibition of the catecholamine oxidation occurs when (τₘₐₓ−τ)=(τ−τ₀). The antioxidant concentration at the half-maximal inhibition ratio is the ratio of the amount of complement to the catecholamine that reduces the rate of catecholamine oxidation by one-half of the maximal reduction in catecholamine oxidation. In a preferred embodiment of this invention, the dosage forms of this invention comprise a concentration of complement at least about 2, preferably at least about 10, preferably at least about 25, preferably at least about 50, preferably at least about 100, preferably at least about 150 times, times higher than the preservative level of complement compound as determined by the at the half-maximal inhibition ratio as determined by the Antioxidant Effect Method. Preferably, the concentration of complement is at least about 2, at least about 10, preferably at least about 25, preferably at least about 50, preferably at least about 100, preferably at least about 150 times higher, preferably at least about 200, times higher than the antioxidant concentration at the half-maximal inhibition ratio determined by the Antioxidant Effect Method.

In some preferred embodiments, the amount of ascorbate compounds used is preferably from about 10 micromolar to 10 millimolar, more preferably from about 100 micromolar to 1 about millimolar for aqueous solutions and suspensions. For compositions comprising epinephrine, preferred compositions comprise from about 1.0 mg to about 1.0 gram of ascorbate per milligram of catecholamine, more preferably from about 10.0 mg to about 100.0 mg ascorbate per milligram of catecholamine. For oral dosage forms, the compositions of this invention deliver from about 500 mg and 5 grams of ascorbate per day. For compositions comprising an opioid, low levels of opioid are preferably used to avoid systemic effects. Preferably, the compositions of this invention deliver from about 0.01 mg/70 kg of body weight to about 1.0 mg/70 kg of body weight per day in solutions of from about 0.01 to about 1.0 mg/ml solutions or suspensions, or in pills, inhalant, or other solid forms comprised of less than 1 mg/daily dose. Preferably the levels of opioid are subefficacious. A “subefficacious amount” of an opioid is an amount which is safe when administered to a human or other animal subject in a composition or method of this invention, but does not create a clinically significant narcotic effect. For compositions comprising a polycarboxylic acid chelater, compositions preferably comprise solutions of from about 1.0 micromolar to about 100.0 micromolar concentration, more preferably from about 5.0 to about 20.0 micromolar concentrations. Such compositions are administered at no more than a total of 1.5 mg/dose or 1.5 mg/minute (during infusion or i.v. drip, etc.) and preferably at less than 0.15 mg/dose or 0.15 mg/minute.

Dosage Forms and Optional Materials:

The compositions of this invention may be in any of a variety of forms, suitable (for example) for oral, rectal, topical or parenteral administration. Depending upon the particular route of administration desired, a variety of pharmaceutically-acceptable carriers well-known in the art may be used. These include solid or liquid fillers, diluents, hydrotropes, surface-active agents, and encapsulating substances. Optional pharmaceutically-active materials may be included, which do not substantially interfere with the activity of the adrenergic compounds. The amount of carrier employed in conjunction with the adrenergic and complement compounds is sufficient to provide a practical quantity of material for administration per unit dose. Techniques and compositions for making dosage forms useful in the methods of this invention are described in the following references, all incorporated by reference herein: 7 Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976); and U.S. Pat. No. 5,646,139, White et al., issued Jul. 8, 1997.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight by the total composition.

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents. Preferred carriers for oral administration include gelatin, propylene glycol, cottonseed oil and sesame oil.

The compositions of this invention can also be administered topically to a subject, i.e., by the direct laying on or spreading of the composition on the epidermal or epithelial tissue of the subject. Such compositions include, for example, lotions, creams, solutions, gels and solids, and may, for example, be locally or systemically administered transdermally or by intranasal, pulmonary (e.g., by intrabronchial inhalation), ocular, or other mucosal delivery. Suitable carriers for topical administration on skin preferably remain in place on the skin as a continuous film, and resist being removed by perspiration or immersion in water. Generally, the carrier is organic in nature and capable of having dispersed or dissolved therein the adrenergic and complement compounds. The carrier may include pharmaceutically-acceptable emollients, emulsifiers, thickening agents, and solvents.

Formulations suitable for mucosal administration by inhalation include compositions of the adrenergic and complement compounds in a form that can be dispensed by inhalation devices among those known in the art. Such formulations preferably comprise liquid or powdered compositions suitable for nebulization and intrabronchial use, or aerosol compositions administered via an aerosol unit dispensing metered doses. Suitable liquid compositions comprise the active ingredient in an aqueous, pharmaceutically acceptable inhalant solvent, e.g., isotonic saline or bacteriostatic water. The solutions are administered by means of a pump or squeeze-actuated nebulized spray dispenser, or by any other conventional means for causing or enabling the requisite dosage amount of the liquid composition to be inhaled into the lungs.

Suitable powder compositions include, by way of illustration, powdered preparations of the active ingredients thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. Aerosol formulations preferably include propellants, surfactants and co-solvents and may be filled into conventional aerosol containers that are closed by a suitable metering valve.

Methods of Treatment

This invention also provides methods of treating disorders associated with the regulation of an adrenergic receptor. Methods of this invention include those comprising:

(a) administering a low dose of an adrenergic compound, and

(b) administering a safe and effective of a complement to said adrenergic compound.

Other methods of this invention comprise:

(a) administering to a subject a safe and effective amount of an adrenergic compound; and

(b) administering to a subject a complement to said adrenergic compound, selected from the group consisting of a hyperpreserving amount of an ascorbate, a safe and effective amount of an opioid, a hyperpreserving amount of a polycarboxylic acid chelater, and mixtures thereof.

The adrenergic compound and the complement compound can be administered concomitantly, or separately. Preferably the adrenergic and complement compounds are administered in a dosage regimen that results in efficacious levels of the compounds in the tissues that are to be treated throughout the desired duration of treatment. Preferably the adrenergic and complement compounds are administered within one hour of each other, more preferably within ten minutes, more preferably at the same time.

The adrenergic compounds and complements of this invention can be administered topically or systemically. Systemic application includes any method of introducing the compounds into the tissues of the body, e.g. intrathecal, epidural, caudal, intramuscular, transdermal, intra-arterial, intra-cardiac, intravenous, intraperitoneal, subcutaneous, sublingual, rectal, nasal, pulmonary, and oral administration. The specific dosage of compounds to be administered, as well as the duration of treatment, are mutually dependent. The dosage and treatment regimen will also depend upon such factors as the specific compound used, the ability of the compound to reach therapeutic concentrations at the site of the action, the nature and extent of other disorders (if any), the personal attributes of the subject (such as weight), compliance with the treatment regimen, the nature of concomitant therapies (if any), and the presence and severity of any side effects of the treatment.

A “low dose” of a given adrenergic compound is from 1% to about 90%, preferably from about 10% to about 50%, of the uncomplemented clinically effective dose of said adrenergic compound that would be administered to a human or other animal subject over a given period of time to obtain a given level of effect. The methods of this invention can be effected by the administration of the adrenergic compound at levels lower than practiced in the art, by administering the adrenergic compound at dosage frequencies longer than practiced in the art, or both. The methods of this invention preferably use less drug to get the same (or greater) effect over the same (or greater) period of time; provide a greater effect using the same (or less) amount of drug over the same (or less) period of time; or afford longer duration of efficacy at the same (or greater) effect using the same (or less) amount of drug. Accordingly, the methods of this invention include methods wherein the compositions of this invention are administered in a number of doses equivalent to the number of doses of an adrenergic compound used in the art, but a reduced dosage levels. Methods of this invention also include methods wherein compositions in the art are administered at the same unit dosage amount, but with reduced frequency. In a preferred embodiment, such dosage levels and regimens are determined using the methodologies described above regarding the compositions of this invention, including the Adrenergic/Complement Dosage Level Determination Method. Preferably, the total amount of adrenergic compound administered according to this invention during a given period of time is equal to from about 1% to about 90%, preferably from about 10% to about 50%, of the product of the number of doses of the drug administered in the art, multiplied by the amount of adrenergic compound administered in each dose in the art, during the given period of time.

The methods of this invention involve administration of an adrenergic compound and a complement to the adrenergic compound to a human or other animal subject for the treatment or prevention of any disorder which is mediated by an alpha- or beta-receptor. Such methods include, without limitation, those which have an affect on blood pressure, the vascular system, the heart, smooth muscles, or metabolism. Such neurological disorders include schizophrenia, Parkinson's disease and attention-deficit hyperactivity disorder. Cardiac disorders include hypotension, forward failure, backward failure and congestive heart failure. Vascular disorders include shock, hypotension, hemorrhage, and disorders associated with anesthesia. Respiratory disorders include nasal congestion, oral and nasal inflammation and swelling (such as caused by cold or flu), chronic obstructive pulmonary disease, asthma, emphysema, and bronchospasm. Gastrointestinal disorders include colic and Crohn's disease. Other disorders and uses include anaphylaxis, interstitial cystitis, overactive bladder syndrome, premature labor, myethsenia gravis, glaucoma, dilation of pupils, and weight reduction.

The compositions and methods of this invention also include the administration of an adrenergic compound to cause homeostasis for topical anesthetics, increasing the duration of anesthetic action. Such anesthetics are administered, for example, by intramuscular injection during dental procedures or skin surgery. Accordingly, such compositions and methods of this invention additionally comprise a safe and effective amount of an anesthetic agent such as lidocaine or procaine. In a preferred embodiment, the level of the anesthetic agent is administered in a lower dose, with less volume of material injected, yielding an equivalent level and duration of anesthesia as conventional compositions that do not contain a complement.

The present invention also provides methods of determining a regimen for regulating an adrenergic receptor in human or other animal subjects, comprising:

(a) selecting an adrenergic compound useful for regulating said receptor;

(b) selecting a complement to said adrenergic compound;

(c) determining the dosage level and frequency of dosing of said adrenergic compound for use in regulating said receptor when administered to said subjects in the absence of said complement;

(d) evaluating the effectiveness of said adrenergic compound in regulating said receptor when administered to said subjects in the presence of said complement, as a function of the dosage level of said adrenergic compound and the dosage level of said complement; and

(e) determining a regimen for regulating said receptor in said subjects by

(i) selecting a dose level of said adrenergic compound which is determined to be effective in said evaluating step (d) and that is lower than the dosage level determined in said step (c);

(ii) selecting a dosage frequency that is determined to be effective in said evaluating step (d) and is longer than the dosage frequency determined in said step (c), or

(iii) both (i) and (ii).

Preferably, selecting step (b) comprises identifying the complement using the physical, chemical or immological techniques described above regarding complement binding. Preferably, step (c) for determining the dosage level and frequency of dosing in the absence of the complement is performed as discussed above regarding the uncomplemented clinically efficacious amount of adrenergic compound. As used herein, the “absence” of the complement refers to levels of complement at the site of action of the adrenergic compound that are not significant, preferably no higher than those associated with typical dietary levels of such complements. Preferably, step (d) for evaluating the effectiveness in the presence of said complement is performed as discussed above regarding the subefficacious levels adrenergic compound in the dosage forms of this invention. As used herein, the “presence” of the complement refers to concurrent presence of the adrenergic compound and the complement at the site of action of the adrenergic compound. In one embodiment of this invention, these steps are performed using the Adrenergic/Complement Dosage Level Determination Method set forth above. Plots such as those set forth in FIGS. 1 and 2 are preferably used in these methods, as discussed above.

The following non-limiting examples illustrate the compositions and methods of the present invention.

### EXAMPLE 1

A patient presents with asthma complicated by degenerative heart disease and has variously used 0.25% or 1% isoproterenol aerosols, 0.10-0.20 mg epinephrine-bitartrate inhalers, and 90 μg albuterol every day at 4-6 hour intervals. The patient has unfortunately experienced unacceptable side effects under all of these therapies, including high blood pressure, palpitations and nervousness from these prior treatments, and is at risk of heart attack. The subject is administered an aerosol composition comprising 0.075% isoproterenol and 1.0% ascorbic acid. The subject is able to control his asthma, with substantially complete symptom relief, using the aerosol at over eight hour intervals. Systemic uptake of the isoproterenol is decreased, thereby eliminating the adverse side effects experienced previously by the patient.

In the above Example, the amount of isoproterenol is decreased to about 0.05%, with substantially similar results. Also in the above Example, the amount of ascorbic acid is increased to about 2.0%, with substantially similar results.

### EXAMPLE 2

A patient presents with asthma and has previously used 90 μg albuterol every day at 4-6 hour intervals. However, his asthma is still not well controlled, as he sometimes fails to get complete symptomatic relief. The subject is administered composition comprising 50 μg albuterol and 0.2 mμg morphine sulfate. The subject is able to control his asthma, with substantially complete symptom relief, taking the composition at six hour intervals. Moreover, the subject experiences no adverse side effects.

In the above Example, the amount of albuterol is decreased to about 10 μg with substantially similar results. Also in the above Example, the amount of morphine sulfate is decreased to about 0.1 mg, with substantially similar results.

### EXAMPLE 3

A patient presents with asthma and has previously used a 0.20 mg/dose epinephrine bitartrate inhaler every day at 6 hour intervals. The subject is administered a composition delivering a unit dose comprising 0.03 mg epinephrine bitatrate and 0.2 mg EDTA from a conventional inhaler. The subject is able to control his asthma, with substantially complete symptom relief, taking the composition at eight hour intervals.

In the above Example, the amount of epinephrine bitartrate is decreased to about 0.10 mg, with substantially similar results. Also in the above Example, the amount of EDTA is decreased to about 0.05 mg, with substantially similar results.

### EXAMPLE 4

A woman presents with glaucoma. She is administered a solution comprising 0.005% epinephrine borate and 10% ascorbic acid in a conventional ophthalmic solution vehicle. (The level of epinephrine in compositions in the art is typically about 0.1-2.0%.) Her glaucoma is controlled, requiring less frequent administration.

### EXAMPLE 5

A patient presents with hypotension. The subject is administered subcutaneously a composition comprising epinephrine as a 1:50,000 sterile aqueous solution of 500 micromolar ascorbate, increasing blood pressure. (Conventional treatment methods involve administration of compositions containing 1:1000 or 1:10,000 epinephrine in sterile aqueous solution, but side effects of these formulations on the patient include fear, anxiety, restlessness, and sleeplessness, dizziness and palpitations. The method of this Example provides equivalent efficacy, increased duration of action, and significantly fewer adverse side effects due to the lower dose of epinephrine.)

### EXAMPLE 6

A patient arrives in the emergency room in full cardiac arrest. The subject is administered, through intracardiac injection, a dose of 0.4 mg epinephrine hydrochloride in a 1 millimolar ascorbate sterile solution. The treatment is repeated twice at 20 to 30 minute intervals, and cardiac rhythm is restored. (Conventional treatment methods involve delivering epinephrine hydrochloride at 0.5 to 1.0 mg intravenously or directly into the heart every five minutes until resuscitation is achieved or the patient is declared moribund.)

### EXAMPLE 7

A patient having muscle weakness accompanying myesthenia gravis is treated by administering intra-arterial delivery of a 4:1,000.000 epinephrine hydrochloride in a sterile aqueous solution of pH corrected 500 micromolar ascorbate. The treatment results in increased muscle power in treated limbs without major side-effects such as increased blood pressure or palpitations. (Conventional treatment methods involve administration of a 1:100,000 sterile aqueous solutions of epinephrine hydrochloride, but with only about 15 minutes of beneficial effect. The method of this Example results in an equivalent level of benefit, but the benefits are extended to last several hours.)

### EXAMPLE 8

A child develops a cold characterized by severe nasal congestion. The subject is administered by nasal delivery a unit dose of a composition comprising 0.005% oxymetazoline hydrochloride in a 1.0 millimolar ascorbic acid solution. The subject's congestion is substantially relieved, over a period of several hours. (Conventional decongestant sprays deliver unit doses of from about 0.025-0.05% oxymetazoline hydrochloride as an active ingredient. The method of this Example affords more powerful, longer lasting activity than the conventional compositions, without use of slow-release compounds.)

### EXAMPLE 9

A subject undergoing surgery for a massive trauma is bleeding uncontrollably at the surgical site. The bleeding is interfering with the visualization and surgical repair of the injury. A solution of 1:10,000 epinephrine and 2.0 millimolar ascorbic acid is topically applied to the site of the bleeding, as a topical hemostat. The bleeding is substantially diminished, with an effect of greater than ten minutes. (Conventional treatment methods comprise administration of 1:1000 to 1:10,000 solutions of epinephrine, but with action of only a few minutes.)

### EXAMPLE 10

A patient presents with Parkinson's disease. The subject is administered an oral composition comprising 50 mg of levodopa and an oral composition comprising 5000 mg of sodium ascorbate. (Conventional treatment methods involve the administration of about 100 to 500 mg of levodopa per day. The method of this Example results in increased brain uptake of levodopa, with greater efficacy and duration of action for each dose, requiring less frequent administration.)

### EXAMPLE 11

A trauma patient arrives at the hospital having lost such a large quantity of blood that she is in hemorrhagic shock. The subject is infused intravenously with 0.50 micrograms of norepinephrine per minute in a 2.0 millimolar ascorbate aqueous or saline solution. (Conventional treatment methods consist of intravenous administration of norepinephrine at a rate of 2 to 4 micrograms per minute. The methods of this Example allow delivery either for a shorter period of time or at a significantly slower rate per minute.)

### EXAMPLE 12

A subject presenting with cardiogenic shock is intravenously administered 0.2 micrograms/kilogram/minute of dopamine in a 0.1 to 2.0 millimolar ascorbate solution, stabilizing the subject. The treatment redistributes body fluids, reestablishes heart function, and increases blood pressure and kidney function. (Conventional treatment methods involve delivering 2.0-5.0 micrograms per kilogram body weight per minute by an intravenous route until the patient is stabilized. The method of this Example increases effectiveness and duration of effect so that less drug is necessary to establish normal functions.)

### EXAMPLE 13

A man presents with typical symptoms of congestive heart failure, including grossly swollen ankles, poor circulation, and inadequate heart function. The subject is administered intravenously 2.0 micrograms of dobutamine hydrochloride per kilogram of body weight per minute in a 0.1 millimolar sterile ascorbate solution. (Conventional treatment methods involve administration of 2.5 to 10.0 micrograms of dobutamine hydrochloride per kilogram of body weight per minute. The methods of this invention provide a more efficacious treatment with greater duration of action. Normal function can be obtained and retained with less drug.)

### EXAMPLE 14

A woman in her fifth month of pregnancy is admitted to the hospital having entered premature labor. The subject is administered a composition of 0.15 mg/ml ritodine hydrochloride and 0.05 mg/ml EDTA, delivered at 0.3 mg ritodine/minute up to a maximum of 0.15 mg/minute for up to twelve hours. Labor contractions are stopped, preventing premature delivery. (Conventional treatment methods include administration of about 0.35 mg/ml of ritodine hydrochloride, albuterol, terbutaline, butaline or fenoterol intravenously at 0.10 mg per minute, slowly increasing the amount by 0.05 mg/minute up to a maximum of 0.35 mg/minute, continuing for at least 12 hours to prevent premature delivery. The method of this Example provides a mixture with enhanced efficacy and duration that can be delivered intravenously at significantly lower doses, One of the primary advantages of these lowered doses of drug is the lower exposure of the fetus to the drug, thus enhancing the safety of the procedure.)

### EXAMPLE 15

A middle-aged lawyer presents with extreme hypertension that is not amenable to dietary salt reduction or the usual set of anti-hypertensive drugs. The subject is administered 0.02-0.1 mg/day of clonidine in a 0.10-1.0 mg/ml solution of EDTA delivered via a transdermal patch, controlling his blood pressure. (Conventional treatment methods include administration of clonidine 0.1-2.4 mg/day delivered via a transdermal patch. The method of this Example provides equivalent efficacy, while reducing side effects such as dry mouth, sedation, sexual dysfunction, and brachycardia.)

### EXAMPLE 16

A heroin addict is admitted to drug treatment with severe withdrawal symptoms, including profuse sweating, extreme nervousness, gastrointestinal distress, and drug craving. The subject is intravenously administered 0.05 mg/day of clonidine a 5.0 millimolar ascorbate solution, effectively eliminating withdrawal symptoms. (Conventional treatment methods include administration of 0.1-0.8 mg of clonidine per day either i.v. or by pill for up to a week. The method of this Example affords treatment of withdrawal symptoms much more effectively and quickly.)

In the above Example, an oral dosage form comprising 0.05 mg and 0.1 grams of ascorbate is substituted for the intravenous solution, with substantially similar results. Also in the above Example, 0.01 mg/kilogram body weight of morphine sulphate can be administered in addition to the ascorbic acid, with substantially similar results. (In this method, the efficacy of the clonidine is increased without providing enough opiate to have any addictive or tolerance producing effects.)

### EXAMPLE 17

A patient presents with pre-cancerous mole on her back, which must be removed. As part of the procedure, a topical anesthetic is injected comprising 0.3 mil of a lidocaine hydrochloride (20 mg/ml) in a sterile saline solution containing 0.0025 mg/ml epinephrine and 0.1 mg/ml EDTA. The presence of the epinephrine causes vasoconstriction at the inoculation site, permitting the anaesthetic to have a longer duration of activity. Effective local anesthesia results, allowing successful completion of the procedure, with no systemic adverse effects from the anesthetic. (A typical treatment might consist of injecting 0.3 mil of a local anaesthetic such as lidocaine hydrochloride in a solution containing 1/50,000 to 1/200,000 epinephrine (g/ml) (or about 0.02 to 0.005 mg/ml). Unfortunately, the amount of epinephrine used in such local injections creates systemic effects such as increased increase blood pressure, heart rate, and nervousness in the patient.)

In the above Example, the EDTA is replaced with ascorbate or morphine sulphate with substantially similar results. Also in the above Example, the level of epinephrine is reduced to about 0.0005 mg/ml, with substantially similar results.

Also in the above Example, the amount of epinephrine in the local anaesthetic is retained at its usual concentration (1/50,000 to 1/200,000), but 1/10,000 (0.1 mg/ml) ascorbate is added. The duration of vasoconstriction (and hence duration of anaesthetic activity) is thereby increased, obviating the need for treating the patient with codeine or other systemic analgesics following the surgery.

Also in the above Example, the composition of the Example is formulated as an opthamic solution, and is used during eye surgery. Similarly, the composition is formulated as an aerosol for nasal inhalation, facilitating nasal examination and surgery. Similarly, the composition is formulated as an ointment for cuts, burns, or other topical applications.

### EXAMPLE 18

A patient is hospitalized for stroke (cortical ischemia). To increase the rate and extent of her recovery, she is administered a single, one-time dose of 0.1 mg/kg D-amphetamine by co-injecting intraperitoneally it with 10 mg/kg ascorbate. Side effects of the treatment are minimal. (A typical treatment would consist of daily injections of 1 mg/kg D-amphetamine for seven days. Side effects often result, including increased heart rate, blood pressure, agitation and sleeplessness.)

In the above Example, the D-amphetamine injection is replaced with a single injection of L-DOPA, 0.1 mg/kg with 10 mg/kg ascorbate, with substantially similar results. (This treatment is an alternative to a conventional injection of 1.0 mg/kg L-DOPA daily, for seven days, without a complement.)

The examples and other embodiments described herein are exemplary and not intended to be limiting in describing the full scope of compositions and methods of this invention. Equivalent changes, modifications and variations of specific embodiments, materials, compositions and methods may be made with substantially similar results.

What is claimed is:

1. A pharmaceutical composition comprising:
(a) a subefficacious amount of an adrenergic compound; and
(b) a safe and effective amount of a complement to said adrenergic compound.

2. A pharmaceutical composition according to claim 1, wherein said adrenergic compound is a catecholamine.

3. A pharmaceutical composition according to claim 2, wherein said catecholamine is selected from the group consisting of albuterol, dopamine, ephedrine, epinephrine, levodopa, norepinephrine, oxymetazoline, phenylephrine, phenylpropanolamine, pseudoephedrine, theophylline, and mixtures thereof.

4. A pharmaceutical composition according to claim 1, wherein said complement is selected from the group consisting of an ascorbate, an opioid, a polycarboxylic acid chelater, and mixtures thereof.

5. A pharmaceutical composition according to claim 4, wherein said complement comprises an ascorbate.

6. A pharmaceutical composition according to claim 5, wherein said ascorbate is ascorbic acid.

7. A pharmaceutical composition according to claim 4, wherein said complement comprises a polycarboxylic acid chelater.

8. A pharmaceutical composition according to claim 7, wherein said complement is EDTA.

9. A pharmaceutical composition according to claim 4, wherein said complement comprises an opioid.

10. A pharmaceutical composition according to claim 1, wherein said opioid is selected from the group consisting of alfentanil, apomorphine, benzomorphan, buprenorphine, butorphanol, codeine, dezocine, dihydrocodeine, dihydrocodeinone, diphenoxylate, Met-enkephalin, Leu-enkephalin, dynorphin A, dynorphin B, fentanyl, heroin, hydrocodone, hydromorphone, kyotorphin, levorphanol, levomethadyl acetate, loperamide, malbuphine, meptazinol, methadone, meperidine, morphiceptin, morphine, nalbuphine, nalmefene, oxymorphone, oxycodone, pentazocine, propoxyphene, sufentanil, and mixtures thereof.

11. A pharmaceutical composition according to claim 4, wherein said composition is suitable for oral administration.

12. A pharmaceutical composition according to claim 11, wherein said complement is an ascorbate.

13. A pharmaceutical composition according to claim 12, wherein said ascorbate is selected from the group consisting of ascorbic acid, sodium ascorbate, calcium ascorbate, dehydrosoascorbic acid, and mixtures thereof.

14. A pharmaceutical composition according to claim 4, wherein said composition is suitable for parenteral administration.

15. A pharmaceutical composition according to claim 14, wherein said complement is an ascorbate.

16. A pharmaceutical composition according to claim 15, wherein said ascorbate is selected from the group consisting of ascorbic acid, sodium ascorbate, calcium ascorbate, dehydrosoascorbic acid, and mixtures thereof.

17. A pharmaceutical composition according to claim 15, wherein said ascorbate is present at a level of from about 0.01 millimolar to about 5 millimolar concentration.

18. A pharmaceutical composition according to claim 14, wherein said complement comprises an opioid.

19. A pharmaceutical composition according to claim 1, wherein said opioid is selected from the group consisting of alfentanil, apomorphine, benzomorphan, buprenorphine, butorphanol, codeine, dezocine, dihydrocodeine, dihydrocodeinone, diphenoxylate, Met-enkephalin, Leu-enkephalin, dynorphin A, dynorphin B, fentanyl, heroin, hydrocodone, hydromorphone, kyotorphin, levorphanol, levomethadyl acetate, loperamide, malbuphine, meptazinol, methadone, meperidine, morphiceptin, morphine, nalbuphine, nalmefene, oxymorphone, oxycodone, pentazocine, propoxyphene, sufentanil, and mixtures thereof.

20. A pharmaceutical composition according to claim 4, wherein said composition is suitable for topical administration.

21. A pharmaceutical composition according to claim 20, wherein said complement is an ascorbate.

22. A pharmaceutical composition according to claim 21, wherein said ascorbate is selected from the group consisting of ascorbic acid, sodium ascorbate, calcium ascorbate, dehydrosoascorbic acid, and mixtures thereof.

23. A pharmaceutical composition according to claim 21, wherein said ascorbate is present at a level of from about 0.01 millimolar to about 5 millimolar concentration.

24. A pharmaceutical composition according to claim 20, wherein said complement is a polycarboxylic acid chelater.

25. A pharmaceutical composition according to claim 24, wherein said complement is EDTA.

26. A pharmaceutical composition according to claim 20, wherein said complement comprises an opioid.

27. A pharmaceutical composition according to claim 26, wherein said opioid is selected from the group consisting of alfentanil, apomorphine, benzomorphan, buprenorphine, butorphanol, codeine, dezocine, dihydrocodeine, dihydrocodeinone, diphenoxylate, Met-enkephalin, Leu-enkephalin, dynorphin A, dynorphin B, fentanyl, heroin, hydrocodone, hydromorphone, kyotorphin, levorphanol, levomethadyl acetate, loperamide, malbuphine, meptazinol, methadone, meperidine, morphiceptin, morphine, nalbuphine, nalmefene, oxymorphone, oxycodone, pentazocine, propoxyphene, sufentanil, and mixtures thereof.

28. A pharmaceutical composition according to claim 20, wherein said administration is transdermal.

29. A pharmaceutical composition according to claim 20, wherein said administration is intranasal or pulmonary.

30. A pharmaceutical composition according to claim 20, wherein said administration is ocular.

31. A pharmaceutical composition according to claim 1, comprising
(a) wherein said adrenergic is a catecholamine; and
(b) a complement is selected from the group consisting of a hyperpreserving amount of an ascorbate, a hyperpreserving amount of a polycarboxylic acid chelater, and mixtures thereof.

32. A pharmaceutical composition comprising:
(a) a safe and effective amount of an adrenergic compound; and
(b) a complement to said adrenergic compound, selected from the group consisting of a hyperpreserving amount of an ascorbate, a safe and effective amount of an opioid, a hyperpreserving amount of a polycarboxylic acid chelater, and mixtures thereof.

33. A composition of claim 32, wherein said catecholamine is a catecholamine selected from the group consisting of albuterol, dopamine, ephedrine, epinephrine, levodopa, norepinephrine, oxymetazoline, phenylephrine, phenylpropanolamine, pseudoephedrine, theophylline, and mixtures thereof.

34. A pharmaceutical composition according to claim 32, wherein said complement comprises an ascorbate.

35. A pharmaceutical composition according to claim 32, wherein said composition is suitable for oral administration.

36. A pharmaceutical composition according to claim 32, wherein said composition is suitable for parenteral administration.

37. A pharmaceutical composition according to claim 32, which is injectable.

38. A pharmaceutical composition according to claim 37, for inducing localized anesthesia, additionally comprising a safe and effective amount of an anesthetic.

39. A pharmaceutical composition according to claim 32, wherein said composition is suitable for topical administration.

40. A pharmaceutical composition according to claim 39, wherein said administration is intranasal or pulmonary.

41. A pharmaceutical composition according to claim 39, wherein said administration is ocular.

42. A method of treating a disorder associated with an adrenergic receptor in a human or other animal subject, comprising:
(a) administering to said subject a low dose of an adrenergic compound; and
(b) administering to said subject a safe and effective amount of a complement to said adrenergic compound.

43. A method of claim 42, wherein said adrenergic compound is a catecholamine selected from the group consisting of albuterol, dopamine, ephedrine, epinephrine, levodopa, norepinephrine, oxymetazoline, phenylephrine, phenylpropanolamine, pseudoephedrine, theophylline, and mixtures thereof.

44. A method of claim 41, wherein said complement is selected from the group consisting of an ascorbate, an opioid, an opiate, a polycarboxylic acid chelater, and mixtures thereof.

45. A method according to claim 44, wherein said complement comprises an ascorbate.

46. A method according to claim 44, wherein said complement comprises a polycarboxylic acid chelater.

47. A method according to claim 44, wherein said complement comprises an opioid.

48. A method according to claim 44, wherein:
(a) said adrenergic compound is a catecholamine; and
(b) a complement is selected from the group consisting of a hyperpreserving amount of an ascorbate, a hyperpreserving amount of a polycarboxylic acid chelater, and mixtures thereof.

49. A method according to claim 41, wherein said administration is oral.

50. A method according to claim 41, wherein said administration is parenteral.

51. A method according to claim 50, wherein said administration is by intramuscular injection.

52. A method according to claim 50, wherein said administration is intravenous.

53. A method according to claim 50, wherein said administration is by subcutaneous injection.

54. A method according to claim 41, wherein said administration is topical.

55. A method according to claim 54, wherein said administration is transdermal.

56. A method according to claim 54, wherein said administration is intranasal or pulmonary.

57. A method according to claim 54, wherein said administration is ocular.

58. A method of claim 41, for the treatment of a neurological disorder.

59. A method of claim 58, wherein said disorder is schizophrenia, or Parkinson's disease.

60. A method of claim 41, wherein said receptor mediates cardiac function.

61. A method of claim 60, for the treatment of hypotension, forward failure, backward failure, or congestive heart failure.

62. A method of claim 41, wherein said receptor mediates smooth muscle function.

63. A method of claim 62, wherein said method potentiates the contraction of vascular smooth muscle tissue.

64. A method of claim 63, for the treatment shock, hypotension, hemorrhage, or disorders associated with anesthesia.

65. A method of claim 63, to cause homeostasis during topical administration of an anesthetic.

66. A method of claim 62, wherein said method potentiates the relaxation of smooth muscle tissue.

67. A method of claim 66, for the treatment of hypertension.

68. A method of claim 41, for the treatment of chronic obstructive pulmonary disease or asthma, emphysema, or bronchospasm.

69. A method of claim 68, for the treatment of asthma.

70. A method of claim 41, for the treatment of colic or Crohn's disease.

71. A method of claim 48, for the treatment of anaphylaxis.

72. A method of claim 41, for the treatment of interstitial cystitis.

73. A method of claim 41, for the treatment of overactive bladder syndrome.

74. A method of claim 41, for the treatment of premature labor.

75. A method of claim 41, for the treatment of myethsenia gravis.

76. A method of claim 41, for the treatment of glaucoma.

77. A method of claim 41, for causing mydriasis for ophthalmic purposes.

78. A method of claim 41, for the treatment of nasal congestion, or oral or nasal inflammation and swelling.

79. A method of treating a disorder associated with an adrenergic receptor in a human or other animal subject, comprising:
(a) administering to said subject a safe and effective amount of an adrenergic compound; and
(b) administering to said subject a complement to said adrenergic compound, selected from the group consisting of a hyperpreserving amount of an ascorbate, a safe and effective amount of an opioid, a hyperpreserving amount of a polycarboxylic acid chelater, and mixtures thereof.

80. A method of claim 79, wherein said adrenergic compound is a catecholamine. selected from the group consisting of albuterol, dopamine, ephedrine, epinephrine, levodopa, norepinephrine, oxymetazoline, phenylephrine, phenylpropanolamine, pseudoephedrine, theophylline, and mixtures thereof.

81. A method according to claim 80, wherein said complement comprises an ascorbate.

82. A method according to claim 79, for the treatment of a neurological disorder.

83. A method of claim 79, wherein said receptor mediates cardiac function.

84. A method of claim 79, wherein said receptor mediates smooth muscle function.

85. A method of claim 84, for the treatment of hypertension.

86. A method of claim 79, for the treatment of asthma.

87. A method of claim 79, for the treatment of nasal congestion, or oral or nasal inflammation and swelling.

88. A method of determining a regimen for regulating an adrenergic receptor in human or other animal subjects, comprising:
(a) selecting an adrenergic compound useful for regulating said receptor; and
(b) selecting a complement to said adrenergic compound;
(c) determining the dosage level and frequency of dosing of said adrenergic compound for use in regulating said receptor when administered to said subjects in the absence of said complement;
(d) evaluating the effectiveness of said adrenergic compound in regulating said receptor when administered to said subjects in the presence of said complement, as a function of the dosage level of said adrenergic compound and the dosage level of said complement; and
(e) determining a regimen for regulating said receptor in said subjects by
(i) selecting a dose level of said adrenergic compound which is determined to be effective in said evaluating step (d) and that is lower than the dosage level determined in said step (c);
(ii) selecting a dosage frequency that is determined to be effective in said evaluating step (d) and is longer than the dosage frequency determined in said step (c), or
(iii) both (i) and (ii).

89. A method according to claim 88, wherein said selecting step (b) comprises identifying said complement by physical, chemical or immunological techniques for detecting binding between said complement and said adrenergic compound.

90. A method according to claim 88, wherein said adrenergic compound is a catecholamine selected from the group consisting of albuterol, dopamine, ephedrine, epinephrine, levodopa, norepinephrine, oxymetazoline, phenylephrine, phenylpropanolamine, pseudoephedrine, theophylline, and mixtures thereof.

91. A method according to claim 88, wherein said complement is selected from the group consisting of an ascorbate, an opioid, a polycarboxylic acid chelater, and mixtures thereof.

92. A method of treating a disorder mediated by an adrenergic receptor using a regimen determined according to the method of claim 88.

93. A method according to claim 88 further comprising the step of identifying a pharmaceutical composition comprising an adrenergic compound and a complement to said adrenergic compound, wherein said composition is effective in regulating said receptor when used in said regimen.

94. A pharmaceutical composition identified according to the method of claim 93.
